(19) United States
(12) Patent Application Publication
(10) Pub. No.: US 2018/0177485 A1
(43) Pub. Date: Jun. 28, 2018

(54) BIOLOGICAL SOUND MEASUREMENT APPARATUS

(71) Applicant: OMRON HEALTHCARE CO., LTD., Muko-shi (JP)

(72) Inventors: Nobuki YAKURA, Kyoto (JP); Makoto Tabata, Kyoto (JP); Kenji Hashino, Kyoto (JP); Takayuki Shiina, Kyoto (JP); Kosuke Inoue, Kyoto (JP); Kei Asai, Kyoto (JP); Shinya Tanaka, Kyoto (JP); Katsumi Murakami, Kyoto (JP); Chizu Habukawa, Kyoto (JP)

(21) Appl. No.: 15/849,825

(22) Filed: Dec. 21, 2017

(30) Foreign Application Priority Data
Dec. 27, 2016 (JP) 2016-254062

(51) Int. Cl. A61B 7/04

(52) U.S. Cl. CPC A61B 7/04

(57) ABSTRACT

A biological sound measurement apparatus includes a head portion including a detection portion that is able to come into contact with a body surface of a biological body and detect a biological sound made by the biological body, and a finger placement portion that a finger can come into contact with and that is located on a side opposite to a pressure receiving surface of the detection portion that comes into contact with the body surface, a main body portion that is gripped by the user, and a connection portion that has flexibility and connects the head portion and the main body portion.

### CROSS REFERENCE TO RELATED APPLICATIONS

This application claims the benefit of priority to Japanese Patent Application No. 2016-254062 filed on Dec. 27, 2016. The entire contents of this application are hereby incorporated herein by reference.

### BACKGROUND OF THE INVENTION

### 1. Field of the Invention

The present invention relates to a biological sound measurement apparatus for measuring a biological sound.

### 2. Description of the Related Art

A biological sound measurement apparatus is used in the diagnosis of illnesses of the circulatory system, the respiratory system, and the like. One type of biological sound measurement apparatus is an electrostethograph that collects biological sound with the use of a microphone. For example, in the electrostethographs described in JP S63-135142A and JP S64-29250A, a chest piece that is constituted by a diaphragm and a bell is provided, vibration sound waves detected by the chest piece are guided to a microphone by a conduit, and the vibration sound waves are converted into an electrical audio signal by the microphone. This electrostethograph is defined by a housing that has a shape suited to gripping and operation by one hand, the chest piece, and the conduit that extends between the housing and the chest piece and guides vibration sound waves detected by the chest piece. Note that the conduit is a hollow three-way tube that is molded from a flexible material.

In recent years, there has been a desire for a biological sound measurement apparatus that can not only be used by medical professionals, but also be used by an ordinary person in the case where the user is the subject, and be used by a parent in the case where their child or infant is the subject. With a biological sound measurement apparatus that can be used by even an ordinary person, in the case where a child has asthma, for example, a parent of that child can use the biological sound measurement apparatus to measure wheezing included in respiratory sound made by the child. However, measuring wheezing requires skilled operation of the biological sound measurement apparatus. For example, when measuring wheezing, the entire surface of the diaphragm needs to be in close contact with the subject's skin, and wheezing cannot be measured if the diaphragm is not pressed parallel against the skin. Also, a large amount of noise is added to the measured sound each time the diaphragm and the skin are brought into and out of contact. A physician continuously adjusts the manner in which the diaphragm is pressed while listening to the measured sound. This skill is based on the experience that the physician has, and it has been difficult for an ordinary person with little experience to measure wheezing with use of a biological sound measurement apparatus.

With the above-described electrostethographs of JP S63-135142A and JP S64-29250A, the housing has a shape suited to gripping and operation by one hand, and the conduit is molded from a flexible material, and therefore the user grips the housing with one hand and can easily change the orientation of the chest piece while touching either the diaphragm or the bell of the chest piece against the body of the subject. For this reason, relatively little skill is needed to operate this electrostethograph. However, in order to measure wheezing included in respiratory sound, it is necessary to finely adjust the orientation of the diaphragm or the bell relative to the body of the subject. Also, in the case where the subject is an infant who is always moving, it is necessary to continuously adjust the orientation of the diaphragm or the bell so as to follow the movement of the infant, and it is difficult for an ordinary person to perform such adjustment when using the electrostethographs described in JP S63-135142A and JP S64-29250A.

Also, the chest piece of the electrostethographs described in JP S63-135142A and JP S64-29250A includes a diaphragm and a bell that face away from each other, and when the user grips the housing with one hand, contact noise or friction noise will be added to the measured sound if the user's finger or the like touches the diaphragm or the bell. For this reason, in order to obtain measured sound that has little noise, the chest piece cannot be operated with a finger or the like.

### SUMMARY OF THE INVENTION

Preferred embodiments of the present invention provide biological sound measurement apparatuses that, regardless of the skill of a user, are able to be adjusted to achieve a state in which biological sound is able to be accurately measured.

A biological sound measurement apparatus according to a preferred embodiment of the present invention includes: a head portion including a detection portion that comes into contact with a body surface of a biological body and detects a biological sound made by the biological body, and a finger placement portion that a finger is able to come into contact with and that is located on a side opposite to a pressure receiving surface of the detection portion that comes into contact with the body surface; a main body portion that is gripped by a user; and a connection portion that has flexibility and connects the head portion and the main body portion.

According to preferred embodiments of the present invention, it is possible to provide biological sound measurement apparatuses that, regardless of the skill of a user, are able to be adjusted to achieve a state in which biological sound is able to be accurately measured.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a diagram showing a case where a biological sound measurement apparatus according to a preferred embodiment of the present invention is used.

FIG. 2 is a side view of a biological sound measurement apparatus according to a preferred embodiment of the present invention.

FIG. 3 is a side view of a state in which the biological sound measurement apparatus shown in FIG. 2 is gripped and operated.

FIG. 4 is a side view including a partial cross-sectional view of a head portion of the biological sound measurement apparatus shown in FIG. 2 when gripped.

FIG. 5 is a side view of a biological sound measurement apparatus according to another preferred embodiment of the present invention.

### DETAILED DESCRIPTION OF THE PREFERRED EMBODIMENTS

Hereinafter, preferred embodiments of the present invention will be described with reference to the drawings.

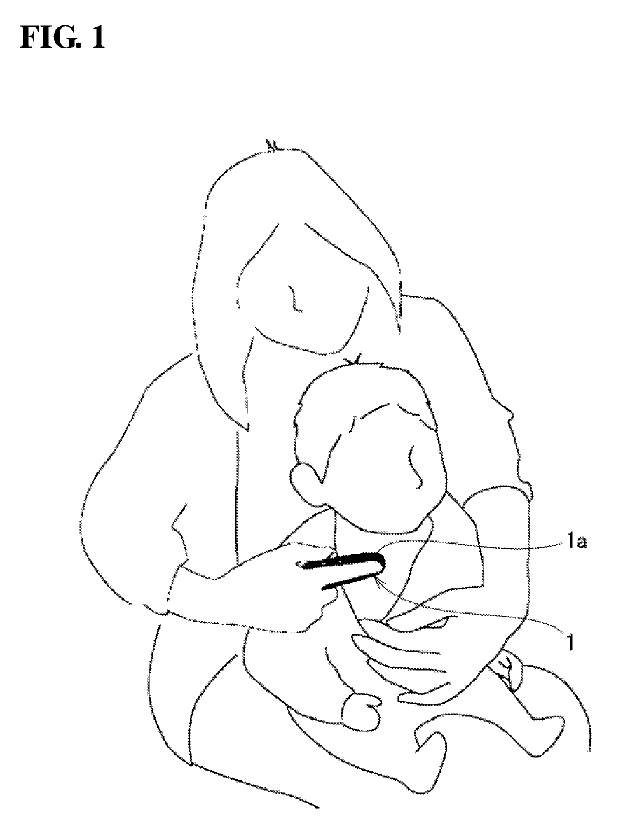

A biological sound measurement apparatus according to a preferred embodiment of the present invention is able to measure respiratory sound made by a subject, by pressing a head portion against a body surface (skin) of the subject. Wheezing is included in respiratory sound made by a person who has asthma, and it is possible to find out the condition of the subject based on wheezing measured by the biological sound measurement apparatus. When measuring respiratory sound made by the subject, as shown in FIG. 1, a user of the biological sound measurement apparatus grips a biological sound measurement apparatus 1 with one hand, and uses a finger to press a head portion 1a against a body surface of the subject (in FIG. 1, an infant). At this time, the user uses their finger to adjust the manner in which the head portion 1a is pressed against the body surface while following movement of the subject. Note that the head portion 1a may be pressed against the body surface by a finger on the hand that is not the hand that grips the biological sound measurement apparatus 1.

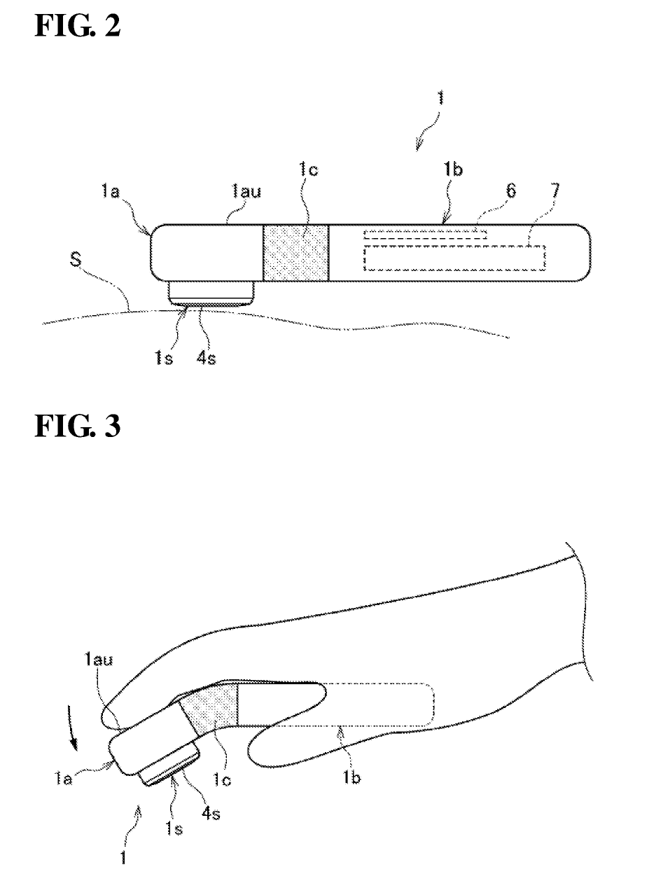

The biological sound measurement apparatus 1 preferably has a size according to which the length in the lengthwise direction fits inside the hand of the user, and the width and thickness are dimensions that enable gripping by an adult hand. FIG. 2 is a side view of the biological sound measurement apparatus 1 according to the present preferred embodiment of the present invention. As shown in FIG. 2, the biological sound measurement apparatus 1 includes the head portion 1a that is pressed against a body surface S of the subject, a main body portion 1b that is gripped by one hand of the user, and a connection portion 1c that has flexibility and connects the head portion 1a and the main body portion 1b. The structure of the biological sound measurement apparatus 1 in the lengthwise direction includes the head portion 1a, the connection portion 1c, and the main body portion 1b in this order. Also, the main body portion 1b preferably has a higher rigidity than the flexible connection portion 1c.

The head portion 1a includes a detection portion 1s that detects wheezing included in respiratory sound made by the subject. The detection portion 1s includes a pressure receiving surface 4s that is pressed against the body surface S of the subject. The pressure receiving surface 4s protrudes in a direction that is orthogonal or approximately orthogonal to the lengthwise direction of the biological sound measurement apparatus 1. Also, the head portion 1a includes a finger placement portion 1au that is located on the side opposite to the pressure receiving surface 4s and is able to be touched by a finger of the user that grips the main body portion 1b.

The main body portion 1b includes internal components such as a controller 6 and a battery 7.

The flexible connection portion 1c preferably has a hollow tubular shape using a material that is capable of elastic deformation, such as silicone, and the head portion and the main body portion 1b are connected to respective ends of the connection portion 1c. A flexible substrate or the like, which electrically connects the detection portion 1s of the head portion 1a and the controller 6 inside the main body portion 1b, passes through the hollow interior of the connection portion 1c. Because the connection portion 1c has flexibility, as shown in FIG. 3, when the user grips the main body portion 1b, places their finger on the finger placement portion 1au of the head portion 1a, and then moves that finger, it is possible to move the head portion 1a toward the palm with the main body portion 1b serving as the fulcrum.

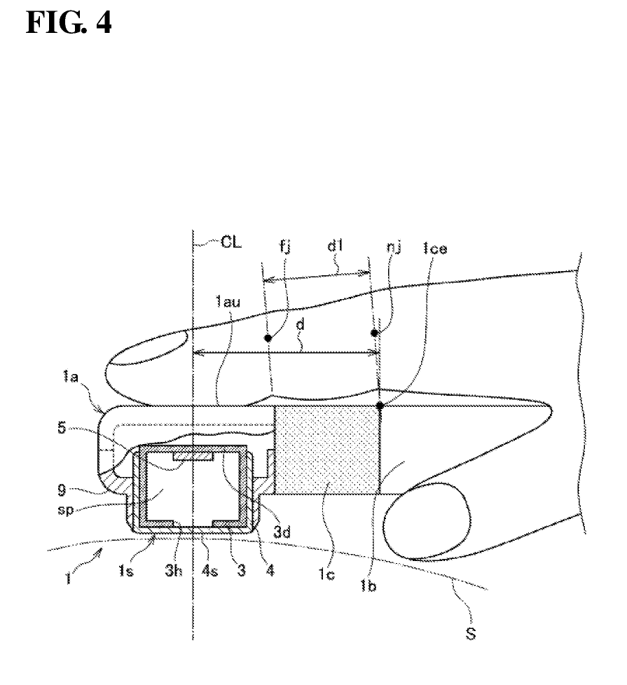

Next, the configuration of the head portion 1a and the positional relationship thereof with the connection portion 1c will be described with reference to FIG. 4. FIG. 4 is a side view including a partial cross-sectional view of the head portion 1a of the biological sound measurement apparatus 1 according to the present preferred embodiment.

As described above, the head portion 1a includes the detection portion 1s that includes the pressure receiving surface 4s that is pressed against the body surface S of the subject. The detection portion 1s includes a housing 3 that is shaped as bottomed hollow cylinder, a housing cover 4, and a microphone 5. The housing 3 is provided to increase sound blocking performance, and is preferably made of a material that has a larger acoustic impedance than air, such as a resin or a metal. An opening 3h provided on one side of the housing 3 is covered by the housing cover 4, which is preferably made of an elastic material such as silicone, and the microphone 5 is provided on a bottom portion 3d of the housing 3 inside a closed space sp that is defined by the housing 3 and the housing cover 4. The detection portion 1s includes wall portions that surround the opening 3h of the housing 3, and is fixed by the wall portions being fitted to an external frame member 9 that is a portion of the housing of the head portion la. The external frame member 9 preferably is a rigid body.

The surface of the housing cover 4 that corresponds to the opening 3h of the housing 3 defines the pressure receiving surface 4s that is pressed against the body surface S of the subject. When the pressure receiving surface 4s is pressed against the body surface S of the subject, vibration sound waves that correspond to subject breathing are transmitted to the closed space sp via the pressure receiving surface 4s of the housing cover 4, and the microphone 5 converts the vibration sound waves in the closed space sp into electrical audio signals.

As shown in FIG. 4, the finger placement portion 1au is located on the external frame member 9 on the surface thereof on the side opposite to the pressure receiving surface 4s, at a position on a center line CL that passes through the center of the pressure receiving surface 4s and is orthogonal or substantially orthogonal to the pressure receiving surface 4s. In the present preferred embodiment, in a side view of the biological sound measurement apparatus 1, a distance d from the center line CL to a line CLp, which is parallel or substantially parallel with the center line CL and passes through an edge portion ice of the connection portion 1c on the main body portion 1b side, preferably is set to about 14.8 mm or higher, for example. The distance d is based on the value of “average value −3σ” regarding a length d1 from a near joint nj to a far joint fj of the second finger (pointer finger) of an adult. Note that the average value (μ) of the length d1 preferably is about 19.9 mm, for example, and these statistics regarding the length d1 are based on “Human Hand Dimensions Data for Ergonomic Design 2010” (published May, 2011) provided by Research Institute of Engineering for Quality Life.

By setting the distance d to the above-described length (greater than or equal to about 14.8 mm, for example), when an adult grips the main body portion 1b with one hand, places the pad of the pointer finger on the finger placement portion 1au of the head portion 1a, and moves the pointer finger so as to mainly bend the near joint nj, the connection portion 1c undergoes elastic deformation with the main body portion 1b serving as the fulcrum, the head portion 1a moves, and the orientation of the pressure receiving surface 4s of the head portion 1a changes. Also, the orientation of the pressure receiving surface 4s is able to be changed in the left-right direction as well, by applying force in the width direction (left-right direction) of the biological sound measurement apparatus 1, which is perpendicular or substantially perpendicular to movement of the pointer finger on the finger placement portion 1au.

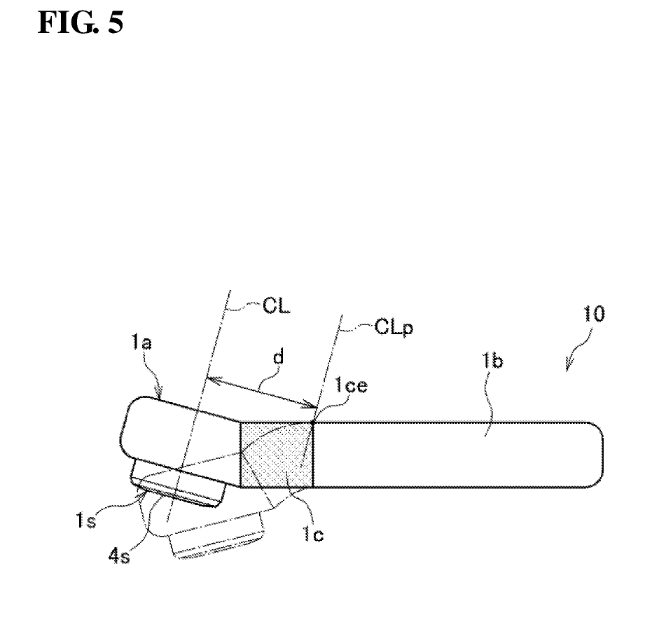

Note that in the example shown in FIGS. 2 to 4, the head portion 1a, the connection portion 1c, and the main body portion 1b are aligned in a straight or substantially straight line when force is not applied to the head portion 1a, but a configuration is possible in which, as shown in FIG. 5, the head portion 1a is inclined toward the finger placement portion 1au side relative to the lengthwise direction of the connection portion 1c and the main body portion 1b. Even with a biological sound measurement apparatus 10 having the configuration shown in FIG. 5, the user can accurately measure respiratory sound with a similar method of use. Note that in the configuration shown in FIG. 5, the edge portion ice of the connection portion 1c that is closest to the main body portion 1b is located on the line CLp that is parallel or substantially parallel with the center line CL and passes through a position that is the distance d away from the center line CL of the pressure receiving surface 4s on the connection portion 1c side.

As described above, in the biological sound measurement apparatus 1 of the present preferred embodiment, the finger placement portion 1au is provided in the head portion 1a, and the connection portion 1c that connects the head portion 1a and the main body portion 1b has flexibility, and therefore when the user of the biological sound measurement apparatus 1 grips the main body portion 1b with one hand and places a finger on the finger placement portion 1au, even if the subject moves when measuring respiratory sound made by the subject, the user is able to change the orientation of the head portion 1a with the finger, or adjust the manner of pressing with the finger. In other words, the user is able to adjust the manner of pressing the head portion 1a with the finger, thus making it possible to easily follow movement of the subject. As a result, even if an ordinary person operates the biological sound measurement apparatus 1, a gap is not provided between the pressure receiving surface 4s of the head portion 1a and the body surface of the subject, and it is possible to accurately measure respiratory sound with a high SN ratio.

Also, in a side view of the biological sound measurement apparatus 1, the distance d, which is the distance from the center line CL of the pressure receiving surface 4s to the line CLp that is parallel or substantially parallel with the center line CL and passes through the edge portion ice of the connection portion 1c on the main body portion 1b side, is set to a size (greater than or equal to about 14.8 mm, for example) that corresponds to the length of the pointer finger of an adult hand, and therefore when an adult grips the main body portion 1b with one hand and places the pad of the pointer finger on the finger placement portion 1au of the head portion 1a, the orientation of the pressure receiving surface 4s of the head portion 1a is able to be easily adjusted by moving the pointer finger. In other words, the head portion 1a is able to be easily operated by the pointer finger.

Also, the external frame member 9 that defines the housing of the head portion 1a, which includes the finger placement portion 1au, is defined by a rigid body, and therefore pressure that the user of the biological sound measurement apparatus 1 applies to the finger placement portion 1au is reliably transmitted to the pressure receiving surface 4s of the head portion 1a. In other words, the user of the biological sound measurement apparatus 1 is able to reliably press the head portion 1a by operating the finger placement portion 1au.

Also, the main body portion 1b has a higher rigidity than the connection portion 1c that has flexibility, and the main body portion 1b is gripped by the user of the biological sound measurement apparatus 1, and therefore the main body portion 1b functions as a fulcrum when the connection portion 1c undergoes elastic deformation due to operation of the head portion 1a. For this reason, it is possible to provide the biological sound measurement apparatus 1 that is easily operable due to the connection portion 1c flexibly deforming in accordance with operation of the head portion 1a. Note that the main body portion 1b does not need to be entirely constituted by a member that has a high rigidity. Specifically, the surface of the main body portion 1b may be covered by a member that has elasticity in order to give a softer feeling when gripped by the user.

Also, it is desirable that the housing 3 that defines the detection portion ls of the head portion 1a is preferably made of a metal that has a high specific gravity. If the housing 3 is made of a metal, the weight of the head portion 1a increases, favorable weight balance is achieved with the main body portion 1b to which the battery 7 is attached, and the user is able to handle the biological sound measurement apparatus 1 with favorable balance. Also, if the housing 3 is made of a metal, it is possible to improve noise reduction inside the closed space sp that is surrounded by the housing 3, thus making it possible to improve the SN ratio of the microphone 5.

The preferred embodiments disclosed above are to be understood as being in all ways exemplary and in no way limiting. The scope of the present invention is defined not by the aforementioned descriptions but by the scope of the appended claims, and all changes which come within the meaning and range of equivalency of the scope of the claims are intended to be included therein as well. For example, the finger placement portion 1au may include a concave surface portion that is concave toward the detection portion 1s side. The concave bend shape of the concave surface portion corresponds to the convex bend shape of the pad of a finger. By providing the finger placement portion 1au with a concave shape, it is possible to increase the area of contact with the finger, and to also improve the fitting sensation between the finger and the head portion 1a. Also, although the biological sound measurement apparatuses of the above preferred embodiments are described as being used to measure respiratory sound made by a subject, it may be used to measure cardiac sound made by a subject or the like.

As described above, the present specification discloses the following matter.

A biological sound measurement apparatus including a head portion including a detection portion that is able to come into contact with a body surface of a biological body and detect a biological sound made by the biological body, and a finger placement portion that a finger can come into contact with and that is located on a side opposite to a pressure receiving surface of the detection portion that comes into contact with the body surface, a main body portion that is gripped by a user, and a connection portion that has flexibility and connects the head portion and the main body portion.

In a biological sound measurement apparatus according to a preferred embodiment of the present invention, a distance from a center line that passes through a center of the pressure receiving surface of the detection portion and is orthogonal or substantially orthogonal to the pressure receiving surface to an edge portion of the connection portion on a main body portion side is preferably about 14.8 mm or higher, for example.

In a biological sound measurement apparatus according to a preferred embodiment of the present invention, the distance is, in a side view of the biological sound measurement apparatus, a distance from the center line to a line that is parallel or substantially parallel with the center line and passes through an edge portion of the connection portion that is closest to the main body portion.

In a biological sound measurement apparatus according to a preferred embodiment of the present invention, a member of the head portion that includes the finger placement portion and surrounds the detection portion is a rigid body.

In a biological sound measurement apparatus according to a preferred embodiment of the present invention, the main body portion has a higher rigidity than the connection portion.

In a biological sound measurement apparatus according to a preferred embodiment of the present invention, a housing of the detection portion is preferably made of a material that has a larger acoustic impedance than air.

In a biological sound measurement apparatus according to a preferred embodiment of the present invention, the housing is made of a metal.

In a biological sound measurement apparatus according to a preferred embodiment of the present invention, the biological sound is wheezing included in respiratory sound made by the biological body.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A biological sound measurement apparatus comprising:
a head portion including a detection portion that is able to come into contact with a body surface of a biological body and detect a biological sound made by the biological body, and a finger placement portion that a finger can come into contact with and that is located on a side opposite to a pressure receiving surface of the detection portion that comes into contact with the body surface;
a main body portion that is able to be gripped by a user; and
a connection portion that has flexibility and connects the head portion and the main body portion.

2. The biological sound measurement apparatus according to claim 1, wherein a distance from a center line that passes through a center of the pressure receiving surface of the detection portion and is orthogonal or substantially orthogonal to the pressure receiving surface to an edge portion of the connection portion on a main body portion side is about 14.8 mm or higher.

3. The biological sound measurement apparatus according to claim 2, wherein the distance is, in a side view of the biological sound measurement apparatus, a distance from the center line to a line that is parallel or substantially parallel with the center line and passes through an edge portion of the connection portion that is closest to the main body portion.

4. The biological sound measurement apparatus according to of claim 1, wherein a member of the head portion that includes the finger placement portion and surrounds the detection portion is a rigid body.

5. The biological sound measurement apparatus according to claim 1, wherein the main body portion has a higher rigidity than the connection portion.

6. The biological sound measurement apparatus according to claim 1, wherein a housing that defines the detection portion is made of a material that has a larger acoustic impedance than air.

7. The biological sound measurement apparatus according to claim 6, wherein the housing is made of a metal.

8. The biological sound measurement apparatus according to claim 1, wherein the biological sound is wheezing included in respiratory sound made by the biological body.
